(19) United States
(12) Patent Application Publication
(10) Pub. No.: US 2022/0219168 A1
(43) Pub. Date: Jul. 14, 2022

(54) MICROGRADIENT-BASED MICROFLUIDIC DEVICES AND HIGH-THROUGHPUT ANALYTICAL METHODS

(71) Applicant: University of Massachusetts, Boston, MA (US)

(72) Inventor: Osman Bilsel, Holden, MA (US)

(21) Appl. No.: 17/607,885

(22) PCT Filed: Jun. 8, 2020
(86) PCT No.: PCT/US20/36591

§ 371 Date: Oct. 31, 2021

Related U.S. Application Data
(60) Provisional application No. 62/859,253, filed on Jun. 10, 2019.

(51) Int. Cl. B01L 3/00

(52) U.S. Cl. CPC B01L 3/502715; B01L 2200/026; B01L 2300/0819; B01L 2400/0472; B01L 2200/0694

(57) ABSTRACT

The invention provides novel microfluidic devices and methods based on microscale gradients that are useful in a variety of applications, such as biomolecule stability, interactions, binding properties, etc.

### PRIORITY CLAIMS AND RELATED APPLICATIONS

This application claims the benefit of priority to U.S. Provisional Application No. 62/859,253, filed Jun. 10, 2019, the entire content of which is incorporated herein by reference for all purposes.

### STATEMENT REGARDING FEDERALLY FUNDED RESEARCH OR DEVELOPMENT

This invention was made with government support under grant number 1353942 awarded by the National Science Foundation, Instrument Development for Biological Research (Type A). The Government has certain rights in the invention.

### TECHNICAL FIELDS OF THE INVENTION

The invention generally relates to microfluidic systems and measurement and analytical methods. More particularly, the invention provides novel microfluidic devices and methods based on microscale gradients that are useful in measuring and analyzing properties and activities of biomolecules such as binding properties, thermodynamic stability, solution properties, etc.

### BACKGROUND OF THE INVENTION

Interaction of proteins with other proteins, RNAs, DNAs and other biomolecules forms the basis of biological machinery. Determination of protein stability, protein-protein interactions, protein-RNA/DNA binding and protein ligand interactions in a quantitative and high-throughput manner is of significant interest to the life science community and pharmaceutical industry. Stability studies, for examples, are crucial during the development of protein and antibody formulations. Stability determination is a significant step in screening biologics and, at a later stage of development, in optimizing biologic formulations for long term storage. The binding affinity of ligands (e.g., small-molecule therapeutics, proteins, nucleic acids) is an important consideration at various stages of candidate screening, lead optimization and formulation development.

Measurement of these biomolecule interactions is often done with some form of multi-sample holder interfaced to a spectrometer. Microwell plates and plate readers are commonly used and available from many vendors. In nearly all cases, a separate sample is prepared in each well (or capillary) for each condition to be tested (e.g., ligand concentration, additive, pH, etc.). Typically, to obtain a binding affinity or stability measurement with some confidence a minimum of 20 different samples need to be prepared and analyzed.

For biologics, such as monoclonal antibodies (mAb), the end user is often interested in a predictor of aggregation propensity. Light scattering techniques such as dynamic light scattering (DLS) are commonly used. DLS is performed at a series of mAb concentrations to predict its aggregation propensity at much higher concentrations and longer timescales (e.g., shelf life). Thermodynamic (DG) and thermal stability (Tm) are also used for this purpose. Because stable proteins are less prone to aggregation, stability measurements (via isothermal stability or temperature melts) may be performed using intrinsic fluorescence. Other techniques such as circular dichroism are also available but less common because they are technically more challenging owing to the need for far-UV light sources, optics and detectors.

These approaches suffer from various limitations. Liquid handling robotics and acoustic droplet-based sample preparation devices have dramatically miniaturized assays by minimizing sample volumes and increasing sample preparation efficiency. However, these devices still rely on the preparation of multiple separate sample wells. One drawback to these approaches is well-to-well concentration variations introduced by liquid handling robotics. Such variations are on the order of a few percent or more. Sample evaporation and adsorption, especially at very low concentrations can also lead to scatter (noise) in a binding curve. Furthermore, obtaining a quantitative binding or stability estimate requires a series of measurements, increasing sample consumption. A further drawback to these approaches is that an accurate binding or stability measurement requires an accurate knowledge of the concentration of the binding-competent protein, which is often known only approximately. Impurities, non-binding modified forms or oligomers often complicate reliable estimation of the actual binding affinity.

Thus, novel and improved devices and methods are desired that address these limitations.

### SUMMARY OF THE INVENTION

The invention provides novel microfluidic devices and methods that overcome many of the limitations in existing devices and techniques, especially in sample requirement and preparation, analytical accuracy and sample throughput. The microfluidic approach disclosed herein focuses on generation of microscale gradients (“microgradients”), a continuum of conditions that can be generated from microliter volume samples, for measuring stability and binding properties of biomolecules.

The microfluidic devices and methods disclosed herein are useful for measuring and analyzing various properties, such as binding affinity, thermodynamic stability and solution properties, of biomolecules such as proteins, antibodies, DNAs, RNAs and other biomolecules. For example, microfluidic devices and methods disclosed herein may be used to quantify the aggregation tendency of biologics such as monoclonal antibodies or to measure ligand-on and off (kₒₙ and k_{off}) rates.

The disclosed invention offers an unconventional approach to measuring a binding curve or a denaturant based stability titration. By relying on diffusion between the two endpoints of a titration a user is able to obtain hundreds of points in a titration using only two samples (the endpoints), each of which will have a volume of a few microliters. Diffusion establishes a microgradient between the endpoints in a laminar flow chip, creating a continuum of concentrations. In this manner, “virtual wells” are created by optical sectioning along the microgradient. Because all of the molecules are in equilibrium, sample-to-sample variations along the “virtual wells” and liquid handling errors are avoided.

In one aspect, the invention generally relates to a microfluidic device or unit, comprising: a first flow channel for flowing a first liquid; a second flow channel for flowing a second liquid; and one or more diffusion channels in fluid communication with the first flow channel at a respective proximal juncture and in fluid communication with the second flow second channel at a respective distal juncture. Each of the first and second flow channels has a width in the range from about 100 μm to about 5 mm. Each of the one or more diffusion channels has a width (or diameter) in the range from about 5 μm to about 100 μm.

In another aspect, the invention generally relates to a high-throughput microfluidic system comprising a plurality of microfluidic devices or units disclosed herein.

In yet another aspect, the invention generally relates to a microfluidic method for measuring or analyzing a biological material. The method comprises: providing a first liquid sample comprising the biological material at a first concentration; providing a second liquid sample comprising the biological material at a second concentration; producing a first flow of the first liquid sample in a first flow channel; producing a second flow of the second liquid sample in a second flow channel; generating one or more microscale gradients in one or more diffusion channels, wherein each of the one or more diffusion channels is in fluid communication with the first flow channel at a respective proximal juncture and in fluid communication with the second flow channel at a respective distal juncture; and measuring one or more properties of the one or more microscale gradients thereby measuring or analyzing one or more properties of the biological material.

In yet another aspect, the invention generally relates to a microfluidic method for generating a microscale gradient between two fluids. The method comprises: providing a first liquid sample; providing a second liquid sample; producing a first flow of the first liquid sample in a first flow channel; producing a second flow of the second liquid sample in a second flow channel; and generating a microscale gradient in a diffusion channel, wherein the diffusion channel is in fluid communication with the first flow channel at a proximal juncture and in fluid communication with the second flow channel at a distal juncture.

In yet another aspect, the invention generally relates to a high-throughput microfluidic method for measuring or analyzing a plurality of biological materials. The method comprises: providing a first set of liquid samples, each comprising one of the plurality of biological materials at a first concentration; providing a second set of liquid samples, each comprising the one of the plurality of biological materials at a second concentration; producing a first set of flows in a first array of flow channels, one for each of the first set of liquid samples; producing a second set of flows in a second array of flow channels, one for each of the second set of liquid samples; generating a plurality of microscale gradients in an array of diffusion channels, wherein each of the array of diffusion channels is in fluid communication with a corresponding flow channel in the first array of flow channels at a respective proximal juncture and in fluid communication with a corresponding flow channel in the array of flow channels at a respective distal juncture; and simultaneously measuring one or more properties of the plurality of microscale gradients thereby measuring or analyzing one or more properties of the biological materials.

### BRIEF DESCRIPTION OF THE DRAWINGS

FIG. 1 shows an overview of spatial manipulation using microfluidics (From Lin and Levchenko, “Spatial Manipulation with Microfluidics” April 2015 Frontiers in Bioengineering and Biotechnology).

FIG. 2 shows a schematic illustration of an exemplary gradient generation device. The flow enters from the left and leaves from the right as indicated by the arrows. The color bar indicates ligand concentration. The gradient is formed in the connector between the two flow channels. These results are from a numerical finite element analysis simulation using the COMSOL software package.

FIG. 3 shows an exemplary flow of protein without ligand (top channel, blue) and protein with ligand (bottom channel, red) is maintained at <1 mm/s flow velocity. A narrow channel connects the two channels. Diffusion is the only transport mechanism in this channel and a gradient is established from ligand concentration [L]=0 to [L]o. The vertical cross section of the diffusion channel is shown on the right, indicating a linear concentration gradient.

FIG. 4 shows an exemplary time-course of the development of a microgradient in a 75 μm×500 μm diffusion channel. The simulations were performed using COMSOL.

FIGS. 5A-5B show an exemplary microfluidic chip design for assuring measurements are recorded on an equilibrated system. Resistors in inlet and outlet are not shown. The rainbow-colored diffusion channel is not drawn to scale in this schematic. The two outlets may be joined as a single common outlet. Bottom panel: an array of gradients for parallelized operation with multiplex detection.

FIG. 6 shows an exemplary 80 μm wide channel with 25 μm constrictions at the connections to the flow channel. The constrictions allow for more flexibility in the flow rates

FIG. 7 shows an exemplary non-linear gradient can be formed from a trapezoidal (or funnel shaped) diffusion channel. This can be utilized for binding studies, which often sample concentration in logarithmic concentration spacing.

FIG. 8 shows an exemplary simulation of the expected improvement in data quality using a microgradient. The microgradient requires an order of magnitude less sample.

FIG. 9 shows a schematic of a prototype instrument. The excitation source would be provided by a compact picosecond diode laser delivering high repetition rate 295 nm excitation of ˜1 mW. Abbreviations: L=lens, F=bandpass filter, PMT=photomultiplier tube. A representative expected binding curve with a large density of excited state lifetime decays detected along the “virtual wells” of the diffusion channel.

FIG. 10 illustrates an advantage of lifetime detection vs steady-state detection. Urea titration of a mAbs. The same data is analyzed using the first moment of the TCSPC decay (top panel) and the total intensity (bottom panel). A more quantifiable transition is obtained using first moment analysis.

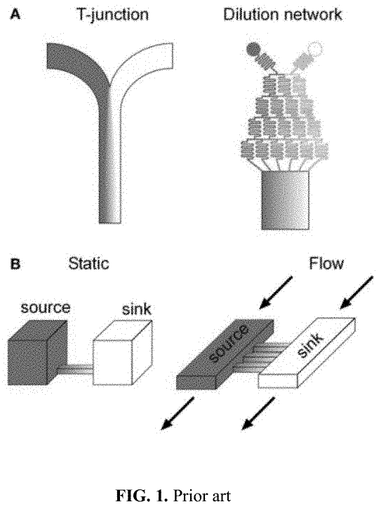

### DETAILED DESCRIPTION OF THE INVENTION

The invention is based in part on the discovery of novel microfluidic devices and methods useful for measuring and analyzing biomolecules such as proteins, antibodies, DNAs, RNAs and other biomolecules. The microfluidic devices and methods of the invention offer significant improvements in sample preparation, analytical accuracy and sample throughput.

The microfluidic devices and methods of the invention utilize microgradients to generate a continuum of sample conditions for measuring biomolecular properties, e.g., protein stabilities and biomolecular binding affinities. The disclosed devices simplify these measurements by requiring only a high concentration and a low concentration microliter scale samples to generate a continuum of concentration gradient for measurement. For stability analysis, the continuum of concentrations may also be denaturant concentrations.

The microgradient approach avoids the need to generate a series of separate samples, which is required by existing techniques. Because all of the measurements are performed on the same gradient continuum in equilibrium the concertation does not fluctuate from point to point, which improves data quantity and quality while requiring less sample. Additionally, the device may include integrated diffusion based laminar mixers for performing dilutions, which allows the user to perform titrations at a continuum of ligand concentrations and several protein concentrations, enabling the protein concentration to be determined accurately from a global analysis of binding curves.

A key feature of the present invention is that by using the endpoint of a titration the microfluidic chip generates a continuum of samples bounded by the endpoints as opposed to individual wells on a microplate. Microgradients are used to prepare “virtual” wells instead of separate wells on a microplate. This has the advantage that the concentration of the ligand being titrated uniformly increases in concentration and will not exhibit well-to-well random variations because the “virtual” wells are in equilibrium with each other. The number of “virtual wells” is limited only by the optical detection method. Any optical detection method can be used. Only small volumes (10 μL or less) of the two endpoints of a titration are required. This conserves on sample and at the same time generates a continuous microgradient. The binding curve or stability curve can be sampled with arbitrary concentration increments. The establishment of equilibrium within the microgradient is based on diffusion and the time evolution of this establishment can be used to estimate the size of the particles (e.g., bound vs unbound) in the microgradient.

The microfluidic devices and methods disclosed herein may be used in a variety of applications, for example, in formulation screening, stability and shelf-life testing for biologics (e.g., proteins and mAb), binding affinity measurements for small-molecule, nuclei acids (e.g., RNA, DNA) and peptide therapeutics in both basic research and drug discovery and development.

In one aspect, the invention generally relates to a microfluidic device or unit, comprising: a first flow channel for flowing a first liquid; a second flow channel for flowing a second liquid; and one or more diffusion channels in fluid communication with the first flow channel at a respective proximal juncture and in fluid communication with the second flow second channel at a respective distal juncture. Each of the first and second flow channels has a width in the range from about 100 μm to about 5 mm (e.g., from about 100 μm to about 2 mm, from about 100 μm to about 1 mm, from about 100 μm to about 500 μm, from about 200 μm to about 5 mm, from about 500 μm to about 5 mm, from about 1 mm to about 5 mm). Each of the one or more diffusion channels has a width in the range from about 5 μm to about 100 μm (e.g., from about 5 μm to about 100 μm, from about 5 μm to about 50 μm, from about 5 μm to about 20 μm, from about 5 μm to about 10 μm, from about 10 μm to about 100 μm, from about 20 μm to about 100 μm, from about 50 μm to about 100 μm).

In certain embodiments, the first and second flow channels have rectangular cross-sectional profiles.

In certain embodiments, the one or more diffusion channels all have rectangular cross-sectional profiles.

In certain embodiments, the one or more diffusion channels all have circular cross-sectional profiles.

In certain embodiments, the microfluidic device or unit has one, two or three diffusion channels. In certain embodiments, the microfluidic device or unit has a single diffusion channel. In certain embodiments, the microfluidic device or unit has two or more diffusion channels.

In certain embodiments of the microfluidic device or unit, at least one of the one or more diffusion channels is characterized by a substantially uniform width from the proximal juncture to the distal juncture.

In certain embodiments of the microfluidic device or unit, each of the one or more diffusion channels is characterized by a substantially uniform width from the proximal juncture to the distal juncture.

In certain embodiments of the microfluidic device or unit, at least one of the one or more diffusion channels is characterized by a non-uniform width from the proximal juncture to the distal juncture.

In certain embodiments of the microfluidic device or unit, at least one of the one or more diffusion channels is characterized by a tapered profile having a gradually reducing width from the proximal juncture to the distal juncture.

In certain embodiments of the microfluidic device or unit, at least one of the one or more diffusion channels further comprises a first constriction at the proximal juncture and/or a second constriction at the distal juncture.

In certain embodiments of the microfluidic device or unit, each of the first and second flow channels has a width in the range from about 250 μm to about 1 mm (e.g., from about 250 μm to about 750 μm, from about 250 μm to about 500 μm, from about 500 μm to about 1 mm, from about 750 μm to about 1 mm).

In certain embodiments of the microfluidic device or unit, each of the one or more diffusion channels has a width in the range from about 25 μm to about 75 μm (e.g., from about 25 μm to about 50 μm, from about 50 μm to about 75 μm).

In certain embodiments of the microfluidic device or unit, each of the one or more diffusion channels has a length in the range from about 250 μm to about 2 mm (e.g., from about 250 μm to about 1.5 mm, from about 250 μm to about 1 mm, from about 250 μm to about 750 μm, from about 500 μm to about 2 mm, from about 1 mm to about 2 mm).

In certain embodiments of the microfluidic device or unit, at least a portion of each of the one or more diffusion channels is optically clear in the range of wavelength from about 180 nm to about 1,000 nm.

In certain embodiments of the microfluidic device or unit, at least a portion of each of the one or more diffusion channels is made of a material selected from quartz, glass, plastic, sapphire, silicon nitride, silicon or diamond. In certain embodiments of the microfluidic device or unit, at least a portion of each of the one or more diffusion channels is made of quartz or glass.

Other window materials may be utilized (e.g., NaCl, KCl, KBr, CaF2, BaF2, MgF2, Csl, KRS-5, AgBr, ZnS, ZnSe, SiO2 and AgCl).

In certain embodiments, the microfluidic device or unit further comprises one or more (e.g., 1, 2, 3, 4 or more) microfluidic pumps (e.g., in fluidic communication with the first channel and/or the second flow channel).

In certain embodiments, the microfluidic device or unit further comprises one or more dilution junctions along the first flow channel and/or one or more dilution junctions along the second flow channel.

The microfluidic device or unit may be a stand-alone device or may be a unit or component of a larger microfluidic system.

In another aspect, the invention generally relates to a high-throughput microfluidic system comprising a plurality of microfluidic devices or units disclosed herein.

In certain embodiments of the high-throughput microfluidic system, the plurality of microfluidic devices or units are arranged in an array. Any suitable number of diffusion channels may be incorporated, for example, about 8 to about 96 (e.g., 8, 16, 32, 64) diffusion channels in total.

The light source may be a laser and a non-laser light source. In certain embodiments, the light source is a laser (e.g., a diode laser, a dye laser or an excimer laser). In certain embodiments, an infrared spectroscopy is coupled with the light source.

In certain embodiments, the high-throughput microfluidic system comprises a diode laser with a light beam in the range of about 180 nm to about 1,000 nm (e.g., about 180 nm to about 800 nm, about 180 nm to about 600 nm, about 180 nm to about 400 nm, about 250 nm to about 800 nm, about 400 nm to about 800 nm, about 600 nm to about 800 nm).

In yet another aspect, the invention generally relates to a microfluidic method for measuring or analyzing a biological material. The method comprises: providing a first liquid sample comprising the biological material at a first concentration; providing a second liquid sample comprising the biological material at a second concentration; producing a first flow of the first liquid sample in a first flow channel; producing a second flow of the second liquid sample in a second flow channel; generating one or more microscale gradients in one or more diffusion channels, wherein each of the one or more diffusion channels is in fluid communication with the first flow channel at a respective proximal juncture and in fluid communication with the second flow channel at a respective distal juncture; and measuring one or more properties of the one or more microscale gradients thereby measuring or analyzing one or more properties of the biological material.

In certain embodiments, one of the first and second concentrations is zero (e.g., a buffer without the biological material).

In certain embodiments, the first flow of the first liquid sample in the first flow channel has a flow rate (i.e., volume/time) substantially the same as the second flow of the second liquid sample in the second flow channel.

In certain embodiments, the first flow of the first liquid sample in the first flow channel has a flow rate substantially different from the second flow of the second liquid sample in the second flow channel.

In certain embodiments, each of the first and second flow channels has a flow rate in the range from about 0.01 μL/min to about 10 μL/min (e.g., from about 0.01 μL/min to about 5 μL/min, from about 0.01 μL/min to about 1 μL/min, from about 0.01 μL/min to about 0.5 μL/min, from about 0.01 μL/min to about 0.1 μL/min, from about 0.05 μ/min to about 10 μL/min, from about 0.1 μL/min to about 10 μL/min, from about 0.5 μL/min to about 10 μL/min, from about 1 μL/min to about 10 μL/min).

In certain embodiments, each of the first and second flow channels has a flow velocity (i.e., distance/time) in the range from about 0.01 mm/s to 10 mm/s (e.g., from about 0.01 mm/s to 5 mm/s, from about 0.01 mm/s to 1 mm/s, from about 0.01 mm/s to 0.5 mm/s, from about 0.01 mm/s to 0.1 mm/s, from about 0.05 mm/s to 10 mm/s, from about 0.1 mm/s to 10 mm/s, from about 0.5 mm/s to 10 mm/s, from about 1 mm/s to 10 mm/s).

In certain embodiments, each of the first and second flow channels has a flow velocity in the range from about 0.1 mm/s to about 1 mm/s (e.g., from about 0.1 mm/s to about 0.5 mm/s, from about 0.5 mm/s to about 1 mm/s).

In certain embodiments, the biological material comprises one or more of cells, polypeptides, nucleic acid molecules, small molecules, nanoparticles, vesicles, micelles, and viruses.

In certain embodiments, the biological material comprises one or more proteins.

In certain embodiments, the biological material comprises one or more antibodies or antibody fragments.

In certain embodiments, the biological material comprises one or more nucleic acid molecules.

In certain embodiments, the biological material comprises at least one protein and at least one nucleic acid molecule.

In certain embodiments, measuring one or more properties of the one or more microscale gradients comprises measuring an optical signal of the one or more microscale gradients.

In certain embodiments, the optical signal comprises one or more of absorption, emission, reflection, light scattering, polarization rotation or optical interference signals.

In certain embodiments, measuring or analyzing a property of a biological material comprises measuring or analyzing qualitative and/or quantitative binding affinity, stability, solubility, diffusion rate, molecular weight, geometric size, conformational dynamics and photophysics (e.g., energy transfer).

In certain embodiments, measuring or analyzing a property of a biological material comprises measuring or analyzing protein-protein interaction.

In certain embodiments, measuring or analyzing a property of a biological material comprises measuring or analyzing protein-nucleic acid interaction.

In certain embodiments, measuring or analyzing a property of a biological material comprises measuring or analyzing aggregation properties of an antibody.

In certain embodiments, measuring or analyzing a property of a biological material comprises measuring or analyzing a ligand-on and off (kₒₙ and k_{off}) rate.

In certain embodiments, the one or more microscale gradients are generated without convection in the one or more diffusion channels.

In yet another aspect, the invention generally relates to a microfluidic method for generating a microscale gradient between two fluids. The method comprises: providing a first liquid sample; providing a second liquid sample; producing a first flow of the first liquid sample in a first flow channel; producing a second flow of the second liquid sample in a second flow channel; and generating a microscale gradient in a diffusion channel, wherein the diffusion channel is in fluid communication with the first flow channel at a proximal juncture and in fluid communication with the second flow channel at a distal juncture.

In certain embodiments of the method, the first liquid sample comprises a first biological material at a first concentration and the second liquid sample comprises a second biological material at a first concentration.

In certain embodiments of the method, the first biological material and the second biological material are the same and the first concentration and the second concentration are different.

In certain embodiments of the method, the first biological material and the second biological material are different.

In certain embodiments of the method, the microscale gradient is generated without convection in the diffusion channel.

In certain embodiments of the method, each of the first and second biological materials comprises one or more of cells, polypeptides, nucleic acid molecules, small molecules, nanoparticles, vesicles, micelles, and viruses.

In yet another aspect, the invention generally relates to a high-throughput microfluidic method for measuring or analyzing a plurality of biological materials. The method comprises: providing a first set of liquid samples, each comprising one of the plurality of biological materials at a first concentration; providing a second set of liquid samples, each comprising the one of the plurality of biological materials at a second concentration; producing a first set of flows in a first array of flow channels, one for each of the first set of liquid samples; producing a second set of flows in a second array of flow channels, one for each of the second set of liquid samples; generating a plurality of microscale gradients in an array of diffusion channels, wherein each of the array of diffusion channels is in fluid communication with a corresponding flow channel in the first array of flow channels at a respective proximal juncture and in fluid communication with a corresponding flow channel in the array of flow channels at a respective distal juncture; and simultaneously measuring one or more properties of the plurality of microscale gradients thereby measuring or analyzing one or more properties of the biological materials.

In certain embodiments of the method, the plurality of biological materials are selected from cells, polypeptides, nucleic acid molecules, small molecules, nanoparticles, vesicles, micelles, and viruses.

In certain embodiments of the method, simultaneously measuring one or more properties of the plurality of microscale gradients comprises measuring an optical signal from the one or more microscale gradients.

In certain embodiments of the method, the optical signal comprises one or more of absorption, emission, reflection, light scattering, polarization rotation or optical interference signals.

In certain embodiments of the method, measuring or analyzing one or more properties of the biological materials comprises measuring or analyzing qualitative and/or quantitative binding affinity, stability, solubility, diffusion rate, molecular weight, geometric size, conformational dynamics and photophysics (e.g., energy transfer).

In certain embodiments of the method, measuring or analyzing one or more properties of the biological materials comprises measuring or analyzing protein-protein interactions.

In certain embodiments of the method, measuring or analyzing one or more properties of the biological materials comprises measuring or analyzing protein-nucleic acid interactions.

In certain embodiments of the method, measuring or analyzing one or more properties of the biological materials comprises measuring or analyzing aggregation properties of an antibody.

In certain embodiments of the method, measuring or analyzing one or more properties of the biological materials comprises measuring or analyzing a ligand-on and off (kₒₙ and k_{off}) rate.

### EXAMPLES

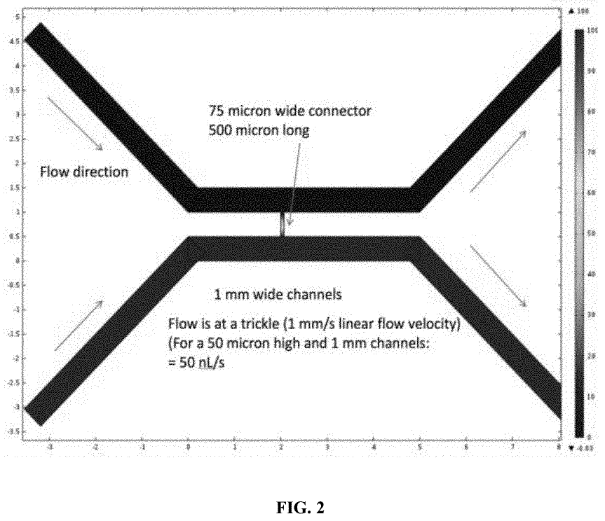

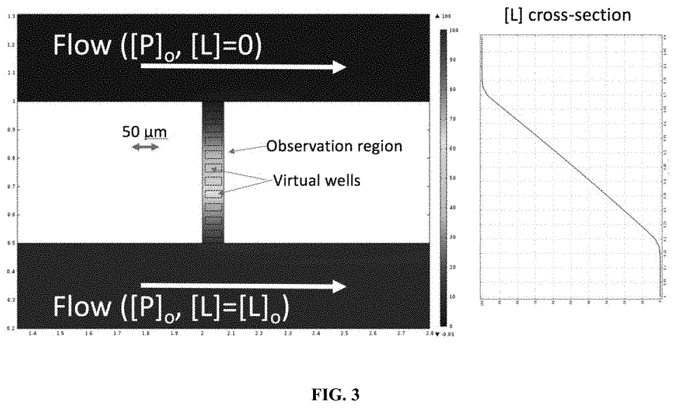

The main principle of the microfluidic device is the generation of a continuum of sample conditions using a microfluidic microgradient. An exemplary design and detailed numerical simulation are shown in FIG. 2 with a zoom into the gradient section shown in FIG. 3. A quantitative cross section plot is shown in FIG. 3 (bottom panel). The color coding in the figures was from numerical simulations that considers both diffusion and convection using the COMSOL Multiphysics package. Under the laminar flow conditions these simulations can accurately represent the concentrations, flow velocities and pressures of an actual device.

In a basic design, a micron scale diffusion (connector) channel connects two flow channels. Because the flows in the two flow channels are maintained to be slow and the diffusion channel very narrow there is no convection in the diffusion channel. The two flow channels are only connected by the diffusion channel in which a microgradient is formed. The microgradient is perfectly linear for solutions with matching diffusion coefficients. For other conditions (e.g., urea or guanidinium chloride gradients) the gradient can be calculated and also calibrated. The microgradient is not critically dependent on the length or the width of the diffusion channel as long as the width of the gradient channel prevents convection. For example, the connection of the diffusion channel to the flow channels can be kept at about 50 μm depending on the applications. An important design factor is that the width of the diffusion channel is much narrower than that of the flow channels.

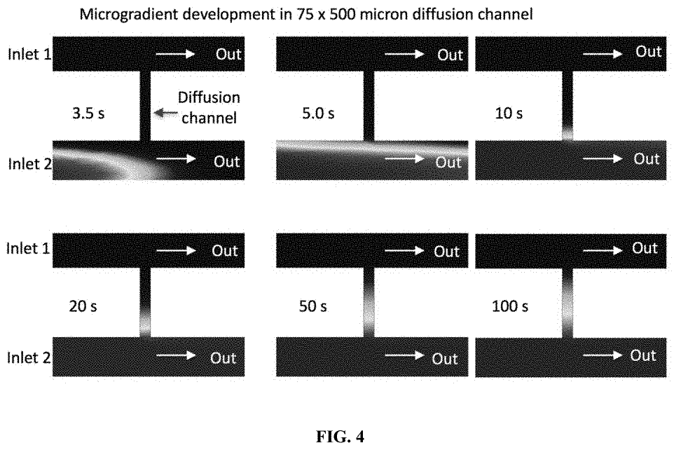

Due to the microfluidic device, the device can be formed with very small (a few microliters) volumes and equilibrium can be established fairly quickly. For example, as shown in FIG. 4, equilibrium can be established in about 2 min for a 500 μm long diffusion channel and even less time is needed for shorter channels.

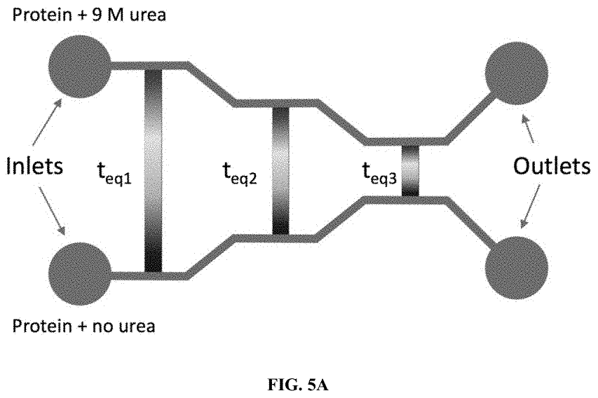

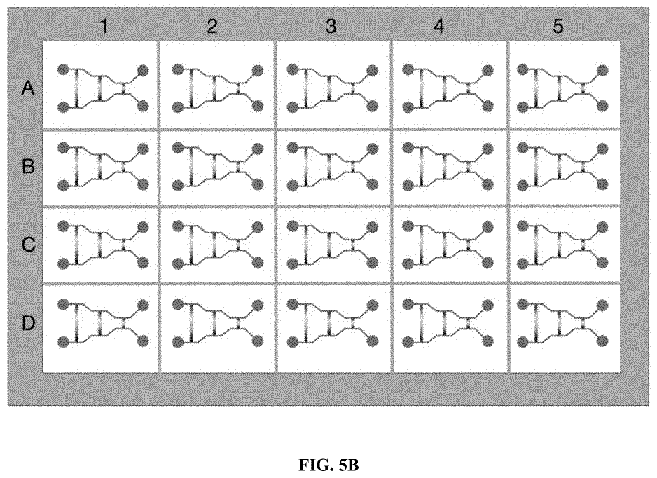

Importantly, multiple microgradients can be setup on a single chip so the user can setup 2, 4, 8, 12, 24 or more of microgradients to monitor binding curves for different ligands. FIG. 5A shows a schematic of how on-chip dilution can be coupled with microgradients to perform a binding curve of protein P with ligand L at multiple concentrations of protein. A combined global analysis gives the true concentration of the binding-competent protein. Experiments at multiple protein concentration are also critical for monitoring aggregation propensity. FIG. 5B shows a multi-sample schematic of a consumable microchip. Note that each titration requires only two inlets of a few microliters. Each titration could also have multiple diffusion channels using the same inlet. The multiple diffusion channels could be different lengths (to test equilibration as shown) and have different geometries (e.g., tapered channels for logarithmic sampling of a binding curve or both a logarithmic and a linear channel). Additional inlets could be added to allow kinetic measurements with different initial conditions.

The microgradient region of the diffusion channels may be scanned or imaged using commonly used spectroscopic techniques.

The microfluidic device and system can be constructed of any suitable materials, e.g., quartz, glass or plastic of various dimensions. For example, the microfluidic device can be used with high numerical aperture imaging systems, light scattering techniques, epifluorescence, absorbance or circular dichroism. The advent of commercially available diode lasers at wavelengths ranging from the far-UV (for circular dichroism) to the near-infrared make a wide range of techniques amenable to this type of approach. The microgradients can also be coupled with surface plasmon resonance (SPR, “Biacore”) to probe binding kinetics for a range of concentrations of ligand simultaneously providing more robust kₒₙ and k_{off} rates through global analysis of multiple protein concentrations.

The microgradient approach disclosed herein offers a number of advantages over the existing devices and techniques.

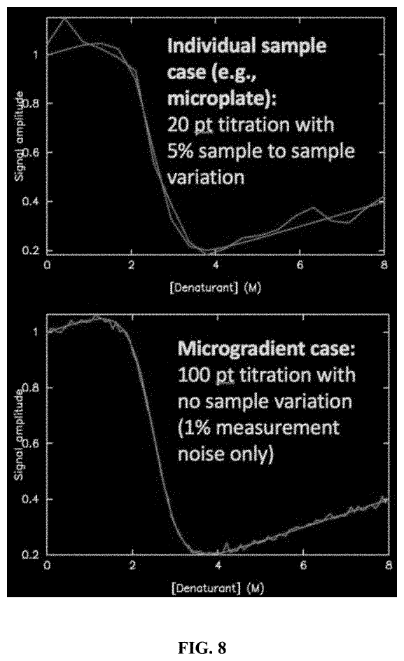

First, there is no sample-to-sample variation in concentration. Because all of the molecules in the diffusion channel microgradient are in equilibrium there is no measurement to measurement fluctuation in protein concentration across the diffusion channel (e.g., only the ligand or denaturant varies in a linear fashion). This avoids the sample delivery, adsorption and evaporation variations from well to well in a microplate. FIG. 8 shows exemplary simulation data traces of a protein stability measurement. In the top panel is what a curve from 20 different samples at increasing denaturant concentrations would look with a 5% pipetting error. In the lower panel, the only error would be the measurement error estimated at 1%. The data density would be an order of magnitude higher with smaller volume requirements.

Second, the disclosed invention drastically reduces sample consumption. For a given analysis, only two microliter volume samples are required to generate the microgradient. As a result, sample consumption is significantly reduced. In a microwell plate format, a separate microliter volume is required for each point in a binding curve or stability curve.

Third, accuracy is significantly improved. Because the microgradient is generated by fundamental physics principles and not by a liquid handler or by manual pipetting, the microgradient is inherently very accurate because manual or machine errors are considerably avoided. Additionally, because of the higher data density and the global analysis of multiple protein concentrations, more accurate binding affinities can be obtained.

In addition, the microgradient approach is compatible with various commonly utilized spectroscopic techniques used, such as fluorescence, absorption, light scattering, etc. Combined thermal and denaturant melts can also be obtained on the same chip to provide better predictors of aggregation propensity for biologics.

Furthermore, the approach may be implemented with multiple titrations on a single microplate-like consumable tray, as schematically shown in FIG. 5A-5B. A device incorporating an array of microgradients can enable a high-throughput measurement and analysis of many samples simultaneously. In certain designs, two microfluidic pumps are sufficient for controlling the flow for all of the channels via a distribution valve. In addition, multiple protein concentrations can be achieved by on chip dilution. A partial schematic of this design is shown in FIG. 5A-5B.

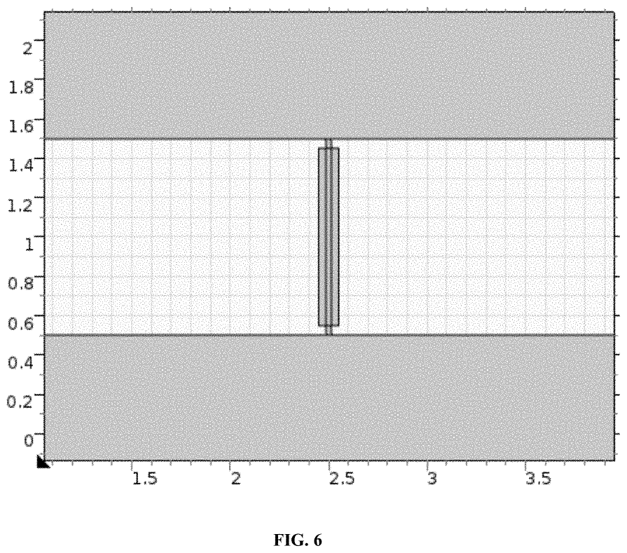

A further important advantage is the built-in flow flexibility of the microgradient approach, which allows modifications of the various components of the device to suit particular needs. For example, constrictions can be placed at the connections of the diffusion channel to the flow channels as schematically illustrated in FIG. 6, which shows an 80 μm-wide channel with constrictions at the connections to the flow channel. The constrictions allow for more flexibility in the flow rates and facilitate measurements in the microgradient region because a very tight focus would no longer be required. A conventional 50 mm plano-convex lens and a laser with good mode quality can readily focus down to about 10 μm FWHM.

The constrictions allow additional flexibility in the flow rate. For example, the flow rates of the two flow channels do not need to be identical as long as the diffusion channel has much higher resistance than the flow channels. In exemplary simulations, a flowrate difference of two-fold did not affect the gradient profile as long as there is no convection in the diffusion channel. This further reduces the sample consumption as the flow rate can be on the order of tens of microns per second, which practically translates to tens of nL per minute.

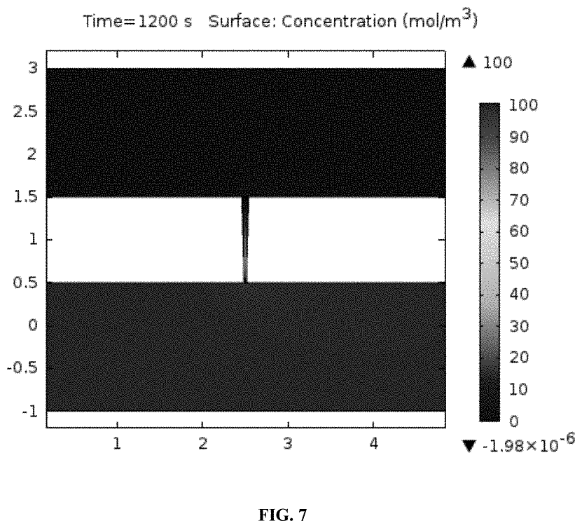

Another variation in the microfluidic design is a tapered diffusion channel, as schematically shown in FIG. 7. For binding curves the optimal spacing for ligand concentrations is often not linear. In the microgradient device one may sample the diffusion channel in a non-linear fashion. However, one can additionally generate a non-linear gradient to enable better resolution of the low concentration region. FIG. 7 illustrates a tapered diffusion channel that can be custom made for a particular application and multiple diffusion channels, e.g., both linear and various non-linear ones, can be placed between the flow channels. Since there is negligible dilution of the flow channel so placing multiple diffusion channels does not require additional inlets.

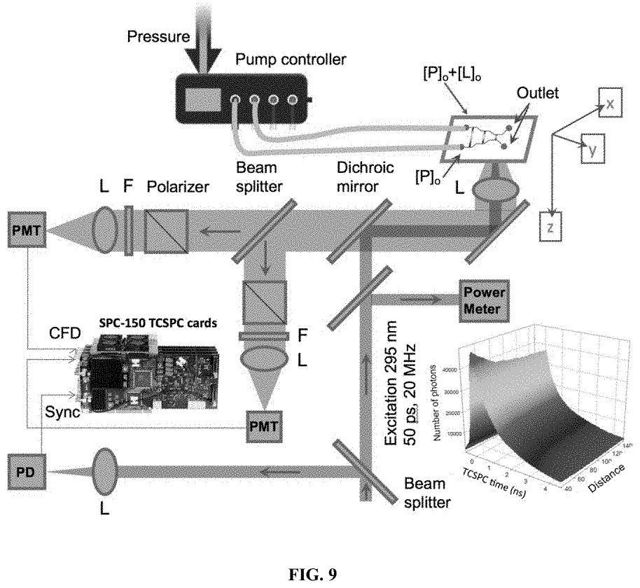

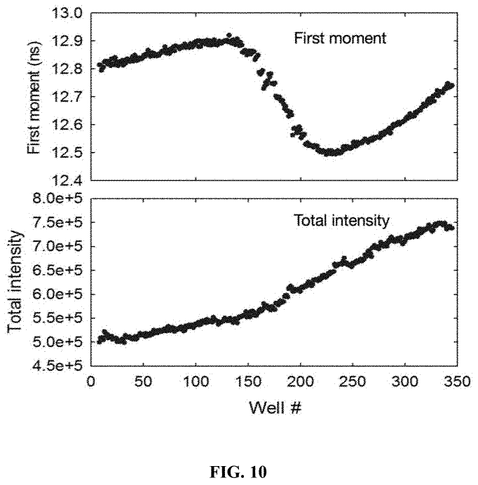

In certain embodiments, the instrument includes an integrated detection system for imaging the diffusion channels. Considering that label-free detection with the highest available sensitivity is a high priority intrinsic fluorescence of tryptophan residues using time-correlated single photon (TCSPC) counting is utilized for as the optical readout. TCSPC can record fluorescence lifetimes with a precision of a few picoseconds in few seconds. The S/N of the average lifetime is proportional to N1/2, where is the total number of photons detected. Additionally, the fluorescence of lifetime of tryptophan (Trp) is very sensitive to the local environment. A schematic of an exemplary embodiment of the instrument is shown in FIG. 9. The microfluidic chip is mounted on a high-speed translation stage with micron-level accuracy. The detection system is essentially a point scanning epi-fluorescence microscope with dual-channel TCSPC detection. The data set is multi-dimensional (lower right), with a time-resolved fluorescence decay acquired at each position along the diffusion channel. Multiple wavelength bandpass filter-based detection is available. The setup can also be used for anisotropy measurements.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A microfluidic device or unit, comprising:
a first flow channel for flowing a first liquid;
a second flow channel for flowing a second liquid; and
one or more diffusion channels in fluid communication with the first flow channel at a respective proximal juncture and in fluid communication with the second flow second channel at a respective distal juncture;
wherein
each of the first and second flow channels has a width in the range from about 100 μm to about 5 mm, and
each of the one or more diffusion channels has a width or diameter in the range from about 5 μm to about 100 μm.

2. The microfluidic device or unit of claim 1, having one, two or three diffusion channels.

3. The microfluidic device or unit of claim 1, having a single diffusion channel.

4. The microfluidic device or unit of claim 1, wherein at least one of the one or more diffusion channels is characterized by a substantially uniform width or diameter from the proximal juncture to the distal juncture.

5. The microfluidic device or unit of claim 1, wherein each of the one or more diffusion channels is characterized by a substantially uniform width or diameter from the proximal juncture to the distal juncture.

6. The microfluidic device or unit of claim 1, wherein at least one of the one or more diffusion channels is characterized by a non-uniform width or diameter from the proximal juncture to the distal juncture.

7. The microfluidic device or unit of claim 6, wherein at least one of the one or more diffusion channels is characterized by a tapered profile having a gradually reducing width or diameter from the proximal juncture to the distal juncture.

8. The microfluidic device or unit of claim 1, wherein at least one of the one or more diffusion channels further comprises a first constriction at the proximal juncture and/or a second constriction at the distal juncture.

9. The microfluidic device or unit of claim 1, wherein each of the first and second flow channels has a width in the range from about 250 μm to about 1 mm.

10. The microfluidic device or unit of claim 1, wherein each of the one or more diffusion channels has a width or diameter in the range from about 25 μm to about 75 μm.

11. The microfluidic device or unit of claim 1, wherein each of the one or more diffusion channels has a length in the range from about 250 μm to about 2 mm.

12. The microfluidic device or unit of claim 1, wherein at least a portion of each of the one or more diffusion channels is optically clear in the range of wavelength from about 180 nm to about 1,000 nm.

13. The microfluidic device or unit of claim 1, wherein at least a portion of each of the one or more diffusion channels is made of a material selected from quartz, glass, plastic, sapphire, silicon nitride, silicon or diamond.

14. The microfluidic device or unit of claim 1, wherein at least a portion of each of the one or more diffusion channels is made of a material selected from NaCl, KCl, KBr, CaF2, BaF2, MgF2, Csl, KRS-5, AgBr, ZnS, ZnSe, SiO2 and AgCl.

15. (canceled)

16. The microfluidic device or unit of claim 1, further comprising one or more microfluidic pumps in fluidic communication with the first channel and/or the second flow channel.

17. The microfluidic device or unit of claim 1, further comprising one or more dilution junctions along the first flow channel and/or one or more dilution junctions along the second flow channel.

18. (canceled)

19. (canceled)

20. A high-throughput microfluidic system, comprising a plurality of microfluidic devices or units of claim 1.

21. The high-throughput microfluidic system of claim 20, wherein the plurality of microfluidic devices or units are arranged in an array.

22. (canceled)

23. (canceled)

24. A microfluidic method for measuring or analyzing a biological material, comprising:
providing a first liquid sample comprising the biological material at a first concentration;
providing a second liquid sample comprising the biological material at a second concentration;
producing a first flow of the first liquid sample in a first flow channel;
producing a second flow of the second liquid sample in a second flow channel;
generating one or more microscale gradients in one or more diffusion channels, wherein each of the one or more diffusion channels is in fluid communication with the first flow channel at a respective proximal juncture and in fluid communication with the second flow channel at a respective distal juncture; and
measuring one or more properties of the one or more microscale gradients thereby measuring or analyzing one or more properties of the biological material.

25-43. (canceled)

44. A microfluidic method for generating a microscale gradient between two fluids, comprising:
providing a first liquid sample;
providing a second liquid sample;
producing a first flow of the first liquid sample in a first flow channel;
producing a second flow of the second liquid sample in a second flow channel; and
generating a microscale gradient in a diffusion channel, wherein the diffusion channel is in fluid communication with the first flow channel at a proximal juncture and in fluid communication with the second flow channel at a distal juncture.

45-58. (canceled)
